(19) United States
(12) Patent Application Publication
(10) Pub. No.: US 2015/0258032 A1
(43) Pub. Date: Sep. 17, 2015

(54) Compositions of Antioxidants and Oil for an antioxidant paste or supplement or liquid or frozen product

(71) Applicant: Kenneth Dale Lightburn, Hartsdale, NY (US)

(72) Inventor: Kenneth Dale Lightburn, Hartsdale, NY (US)

(21) Appl. No.: 14/215,502

(22) Filed: Mar. 17, 2014

(51) Int. Cl. A61K 9/48; A61K 36/87; A61K 36/815; A61K 36/82; A23L 1/30; A61K 36/54; A61K 36/9066; A61K 36/899; A61K 36/258; A61K 36/889; A61K 36/185

(52) U.S. Cl. CPC A61K 9/4875; A61K 36/889; A61K 36/87; A61K 36/815; A61K 36/82; A61K 36/185; A61K 36/54; A61K 36/9066; A61K 36/899; A61K 36/258; A23L 1/3006; A23L 1/3002; A23V 2002/00

(57) ABSTRACT

The invention provides ways to make high antioxidant highly nutritious natural powders appealing and palatable in additives or spreads or drinks or snacks or frozen products or more bio-available in capsules to consumers.

The foods or spreads or drinks or snacks so prepared are functional or fortified and enable a high level of antioxidants to be incorporated in such a way as to make them tasty and enjoyable.

The encapsuled supplements are offered with a natural powder, or any beneficial powder, together with an oil and particularly with a high Medium Chain Triglyceride (MCT) oil so that a simple form of microencapsulation is achieved with the resulting increase in bioavailability and absorption of the antioxidants or beneficial components from the high antioxidant natural and other powders.

This application claims the benefit of provisional patent application Ser. No. 61/78812 filed 2013 Mar. 15, 2013 by the present inventor.

### BACKGROUND

1. Prior Art

The following is a tabulation of some prior art that presently appears relevant:

U.S. Patent Documents

| Patent number | Issue Date | Patentee |
|---|---|---|
| 6,846,501 | Jan. 25, 2005 | Prosise; et al |
| 8,053,007 | Nov. 8, 2011 | Innocenzi; Mark |
| 8,062,688 | Nov. 22, 2011 | Greither; Thomas |
| 8,476,058 | Jul. 2, 2013 | Simon; et al |
| 8,486,899 | Jul. 16, 2013 | Tanaka, et al |

| | | |
|---|---|---|
| 8,507,018 | Aug. 13, 2013 | Chevaux; et al |
| 8,629,175 | Jan. 14, 2014 | Dutta |
| 8,636,985 | Jan. 28, 2014 | Barron |
| 8,642,097 | Feb. 4, 2014 | Maniga |
| 8,668,950 | Mar. 11, 2014 | Wang |
| 8,669,292 | Mar. 11, 2014 | Godin |

US Patent Application Publications

| | | |
|---|---|---|
| 6,1716,462 | Apr. 6, 2004 | Prosise; et al |
| 8,8551,540 | Oct. 8, 2013 | D'Amelio, Sr |

2. Background of the Invention

The field of endeavor is that of diet improvement using a functional food or supplement improvement. This is done through the composition of high antioxidant, high anti-inflammatory natural powders or extracts and flours together with coconut oil or fractions thereof or other oils containing high levels of Medium Chain Triglycerides (MCTs) or MCTs alone and proving this as an addition to other basic foods or as a supplement. Especially together with a bean or pulse or fruit or vegetable paste or compote and perhaps a sweetener.

A variety of foods are needed in a healthy diet to help provide a wide array of micronutrients, macronutrients and non-nutrient plant components (also known as phytochemicals and polyphenols including flavonols. [1], [2], [3], [4] “Functional food” is a term used for any food or beverage designed and/or marketed with a health benefit (physical or emotional), implicit or explicit.[5]

“Functional foods” expands the category that both industry and consumers commonly refer to as “fortified foods.” According to the American Dietetic Association all foods are functional at some physiological level because they provide nutrients or other substances that furnish energy, sustain growth, or maintain/repair vital processes. However, functional foods move beyond these necessary functions to provide additional health benefits that may reduce disease risk and/or promote optimal health. Functional foods include conventional foods, modified foods (i.e., fortified, enriched, or enhanced), medical foods, and foods for special dietary use.[6]

According to The New York Times article, ‘Foods with Benefits, or So They Say’, Functional foods are divided into two categories. Foods with naturally occurring benefits, such as blueberries and red cherries which come loaded with antioxidants called flavonoids; and carrots, spinach, kale and sweet potatoes which are packed with antioxidants [7], and ‘fortified foods’, foods with added health benefits.

Fortified or functional foods can be products that have ingredients added to them to increase their health potential. For example buttery spreads with added Omega-3 may promote better heart health than unfortified butter.[8] Orange juice fortified with calcium and DHA may help fortify bones and increase mental health for children and Heinz ketchup is fortified with lypcopene, a powerful antioxidant.

Sales of functional foods and beverages totaled $37.3 billion in the United States in 2009, up from $28.2 billion in 2005, according to estimates from the Nutrition Business Journal, a market research firm.[9] “The majority of American consumers really believe in the concept that certain foods provide benefits that go beyond basic nutrition or reduce the risk of disease,” says Wendy Reinhardt-Kapsak, the senior director of health and wellness at the International Food Information Council, an industry-financed group specializing in health and nutrition information. [10]

Some of the antioxidant powders that we can add to foods with an oil so as to create functional foods include high antioxidant powders such as, grape seed flour or powder or extract, matcha powder (green tea), goji berries, cocoa dry powder unsweetened, achai fruit pulp/skin powder, raw sumac bran, sorghum bran, ground spices including cloves, oregano, rosemary, thyme, cinnamon, vanilla beans, sage, szechuan pepper, sorghum bran (black), rosehip, Sumac Grain raw, parsley dried and ground, nutmeg basil, pine bark extract.

Antioxidants neutralize free radicals. Free radicals are produced by the normal action of the cells in producing energy and these need to be neutralized. Furthermore, much of the American diet consists of processed foods with an excess of free radicals relative to antioxidants and this total excess results in cellular and DNA damage. Indeed the environment provides many more free radicals, for example the first hand inhalation of the smoke of one cigarette is believed to deliver 1 billion free radicals into the body. Diseases and rapid aging are well recognized as resulting from smoking. By providing a highly palatable food with high antioxidants this imbalance in the normal diet can be potentially offset. The sweetened red bean paste is already regarded as highly palatable.

ORAC or OXYGEN RADICAL ABSORBANCE CAPACITY is a measure of the antioxidant capacity of a food or compound. So called Super Foods are typically those with very high antioxidant values. Antioxidants are essential to optimizing health by helping to combat the free radicals that can damage cellular structures as well as DNA. Blueberries for example are regarded a having very high antioxidant levels. In terms of U.S. fruit consumption, blueberries rank only second to strawberries in popularity of berries. Blueberries are not only popular, but also repeatedly ranked in the U.S. diet as having one of the highest antioxidant capacities among all fruits, vegetables, spices and seasonings. The ORAC value of high antioxidant powders is typically many times that of blueberries and that of the grape seed flour is, when measured in water soluble terms, between 20 and 70 times the ORAC value of blueberries.

Grape seed Flour is made by separation of the seeds during the production of wine or juice from grapes. Subsequently the seeds are stored and then crushed to capture the oil which is separately sold for cooking purposes. The residual power is typically ground and reduced to a size of 300 micron and stored and on demand the 300 micron is variously further ground to finer powders such as 200, 100 and 63 micron powders and smaller.

The use of finely ground flours such as 100 Micron (one micron is one millionth of a meter) which is approximately equivalent to the US measure of 140 mesh, or similar sizes ranging from 300 down to 63 micron or less, in the compositions allows a large surface area for the body's digestive enzymes to attack and digest the antioxidants and the oil suspension allows some of the composition to be carried from the stomach to the small and large intestines for more gradual digestion.

One component, grape seed flour, has uniquely high levels of OPCs (Oligomeric Proanthocyanidins) and these can pass through the blood brain barrier and thus reduce brain inflammation and reduce the formation of beta amyloid plaque which is closely associated with Alzheimer's disease. In addition there are hundreds of research papers regarding both grape seed extract and pine bark extract which contain said OPCs and these mention benefits to many bodily functions and organs. [11] [12]

Grape seed flour or powder has been used in breads and for baking and in food bars for several years in Europe and here but not until now in a paste or liquid or frozen product with a coconut oil or similar base. There is also no record or indication that it has ever further been used in combination with a bean paste or fruit or vegetable compote.

Grape seed power is used in capsules as a supplement offering high OPCs and coconut oil is sold in capsules alone but the two have not been combined, nor has the grape seed extract been combined with coconut oil in a capsule.

Proanthocyanidins refer to a large class of polyphenols called flavanols, in which occur OPC's (oligomeric proanthocyanidins), the simplest flavanols. More complex polyphenols, having the same polymeric building block, form the group of tannins. OPC's were discovered in 1947 by Prof. Jacques Masquelier, who developed and patented techniques for the extraction of oligomeric proanthocyanidins from pine bark and grape seeds [13].

Maritime Pine Bark and grape seeds similarly yield proanthocyanidins under extraction and there is a great deal of research to support a very large number of health benefits from these proanthocyanidins. [11], [12]

Proanthocyanidins have high ORAC and are the principal vasoactive polyphenols in red wine that are linked to a reduced risk of coronary heart disease and to lower overall mortality.[14]

Proanthocyanidins are present at higher concentrations in wines from areas of southwestern France and Sardinia, which are associated with increased longevity in the population. Earlier studies that attributed this health benefit to resveratrol were premature because of the negligible amount of resveratrol in red wine.

Proanthocyanidins suppress production of a protein endolethin-1 that constricts blood vessels.[14]

These studies provide data supporting the French Paradox that hypothesizes that intake of proanthocyanidins and other flavonoids from regular consumption of red wines prevents occurrence of a higher disease rate (particularly of cardiovascular disease and diabetes) in French citizens on high-fat diets.[14]

Proanthocyanidins have antioxidant activity and they play a role in the stabilization of collagen and maintenance of elastin—two critical proteins in connective tissue that support organs, joints, blood vessels, and muscle. Possibly because of their effects on blood vessels, proanthocyanidins have been reported in double-blind research to reduce the duration of edema after face-lift surgery from 15.9 to 11.5 days. In preliminary research, proanthocyanidins were reported to have anti-mutagenic activity (i.e., to prevent chromosomal mutations).

Common antioxidants currently used are vitamin C and vitamin E; however, studies show that proanthocyanidins antioxidant capabilities are 20 times more powerful than vitamin C and 50 times more potent than vitamin E.[15] Proanthocyanidins found in French maritime pine bark and grape seed extract work directly to help strengthen all the blood vessels and improve the delivery of oxygen to the cells. Proanthocyanidins also have an affinity for cell membranes, providing nutritional support to reduce capillary permeability and fragility. Although flavonoids are widespread in nature, the powerful proanthocyanidin compound is most abundant and available from the bark of the maritime pine and in grape seeds, or pips.

In one study on mice, proanthocyanidins may have had antidepressant effects and MAO inhibitory properties.[16]

MAO is an enzyme in the blood stream that causes an increase in the deposition of arterial plaque. It is created by a particular bacteria that reaches significant populations in the gut of a person consuming red meats (including Pork), which have a significant amount of L-Carnitine, on a frequent basis. Infrequent red meat eaters and vegetarians do not develop this high bacterial population or the resulting MAO.[17]

One specific problem that this invention addresses is that chemical extraction processes of the antioxidant powders are potentially going to alter the antioxidants or a reaction may add unwanted chemical compounds to the finished extract. In using a natural powder the antioxidants are made bio-available without chemical extraction by grinding them to a fine powder and then mixing them with coconut oil as a carrier oil. This makes available the antioxidants in the powder without chemical extraction and presents them and at a lower cost.

Another specific problem that this invention addresses is that of adding attractive taste and high antioxidant levels in a gluten free form or to gluten free foods which are often unattractive to taste and low in antioxidants.

It also addresses the problem of taste of coconut oil to many people who would benefit from ingesting the medium chain triglycerides (MCTs) but find the taste of the oil unattractive. Regarding the health benefits of MCTs see Dr Mary Newport's book.[18]

It also addresses the problem of the taste and texture of the Grape Seed Flour and matcha powder and other antioxidant powders in that by themselves they do not have an attractive taste.

It also addresses the problem of introducing high antioxidant high anti-inflammatory food to people with a low intake of such antioxidants by making it highly palatable where used as a food especially when added to a sweetened bean or fruit or vegetable paste.

Some of the antioxidants in these powders are lipid or fat soluble and some are water or hydro soluble. The coconut oil therefore can first dissolve out the lipid soluble antioxidants for prior digestion and then the water soluble antioxidants are extracted by the digestive system from the remaining, now uncovered, particles of powder.

Furthermore it provides a mixture of grape seed extract or grape seed flour together with medium chain triglycerides (MCTs) or oil containing MCTs such as coconut oil so that the capacity of the grape seed extract to prevent plaque formation is added to that of the health benefits of the MCTs in the one supplement and in addition to reduce brain inflammation.[19]

Adzuki beans (red beans) are used extensively in Japanese, Chinese and Korean cultures and to make various textures of red bean paste with the addition of sugar. This paste is used extensively in a variety of sweet dishes encased in rice flour or pastry but none of these dishes include coconut oil or an antioxidant to turn the food into a functional food.

Grape seed extract has a measured ORAC value of approximately 1.2 million (μmol TE/100 g) (micromol Trolox Equivalent per 100 grams) and this is only about seven times as great as that of some grape seed flours. The value of the grape seed flour therefore in a functional food can easily be recognized as it is easy to include seven times as much powder by weight in a food thus delivering the same effect as a supplement.

Another advantage in the processing of these products is that less heat or other sterilization method is required to ensure sterilization of the powders used as the coconut oil has antibacterial and antiviral properties at room temperatures. This lessens the degradation of antioxidants in the powders in processing.

### BRIEF SUMMARY OF THE INVENTION

Unique food combinations for the introduction of high antioxidant and anti-inflammatory flours or powders a spreadable or liquid or frozen form of food or a supplement for people or animals in combination with coconut oil or fractions thereof or alternate high MCT oil.

Recipes for introducing a highly palatable blend consisting largely of a combination of high antioxidant powders or flours (usually strongly anti-inflammatory) such as grape seed flour and/or matcha powder (green tea) powder and/or goji powder and/or Achai and/or Spirulina and/or Pine Bark Extract or other high antioxidant high anti-inflammatory natural powder products, mixed homogenously with a high MCT oil such as coconut oil or warmed or cooked with the oil to thicken and then in some cases added to a bean paste or fruit compote or paste so as to produce a spread or topping or liquid with high antioxidants or as a frozen food for people or animals. A recipe using coconut oil and red bean (Adzuki) or any bean paste and sweetener can increase the palatability of the powder/coconut oil is also unique.

The benefits of high antioxidant, high anti-inflammatory powders in a suspension of such oil is that they are very powerful reducers of free radicals and as such can assist the body in healing itself or prevent damage to the cells and DNA and the coconut oil allow gradual digestion through different parts of the digestive system as the antioxidant is separated from the oil and more fully absorbed rather than the rapid absorption (or destruction of the antioxidant in the stomach acid) if in a water based carrier or without an oil carrier.

Antioxidant compounds are either lipid or water soluble as evidenced by the different ORAC lipid and ORAC hydro values of various high antioxidant powders that are typically added together to give a full picture. The oil therefore begins the extraction of the lipid soluble antioxidants on mixing thus making them more readily available for digestion and furthermore initially protects the particles with their remaining water soluble antioxidants from the stomach acid. This allows a more sequential digestion as the oil is digested away from the particle surface and is no longer a coating for the fine particle. It is in effect a low cost microencapsulation.

The benefits particularly of grape seed flour consumption for example is that it has a high level of OPCs or Oligomeric Proanthocynanidins which can pass through the blood brain barrier and scavenge for free radicals and so improve mental functioning and reduce formation of beta amyloid plaque. Some components of grape seed flour i.e. grape seed extract, have been shown to stop the formation of plaque in the brains of Alzheimer's mice. Benefits of OPC's have been found to be major in terms of anti-inflammatory properties and beneficial to many organs of the body with hundreds of research papers often focused on the same OPC's in French Pine Bark Extract and also on Grape Seed Extract.

The benefits of coconut oil consumption are that the ketone bodies soon converted by the liver of the MCTs (without pre-storage as fat cells) are capable of fueling neurons which can no longer easily take up glucose or of fueling muscle cells. The study and findings by Dr Mary Newport and others has fueled a surge in interest in coconut oil consumption, particularly by those with elderly parents suffering from Alzheimer's disease or other neurological diseases or by anyone threatened with an age related decline in mental abilities. [18]

The benefits of the combination of the coconut oil and the high antioxidant powders are due to the presence of both lipid (fat) soluble and water soluble antioxidants in the powders. By preparing the mixture of the powders and oil the lipid soluble antioxidants are taken into the coconut oil and presented as such to the digestive system. At the same time the water soluble antioxidants remaining in the powder are carried further into the digestive system while temporarily protected by the coconut oil coating, in effect being encapsulated in the coconut oil. This gives the antioxidants two separate pathways for being absorbed into the body and thus increases bioavailability of the antioxidant.

Benefits of a high antioxidant diet are numerous and also include that it can reduce Alzheimer's in 80% of the people with the “Alzheimer's” gene APOE e4 which is associated with Alzheimer's risk. [20],[21],[22].

A further benefit of the combination of the grape seed flour and coconut oil is that the coconut oil has antiviral and antibacterial propertied and this assists in presenting a safer and healthier product.

### DETAILED DESCRIPTION OF THE INVENTION

The ratio by volume of Grape seed Flour or Extract or other high antioxidant powders flours to Coconut oil or high MCT oil ranges from 1 volume of powder to 10 volumes of oil to a ratio of 10 volumes of powder to one volume of oil. The combination is then mixed, and perhaps warmed or cooked, into a homogenous mixture, in some cases to thicken the combination. This can be the fortifying ingredient for a huge range of foods.

A range of spreads or liquids or toppings or supplements can be made using the ratio of this first combination to that of the bean or other type of suitable material (such as fruit compote). The range of combination to suitable material can be between eight volumes of the combination to one of the suitable (suitable in terms of taste and consistency) material up to one volume of the combination to twenty volumes of the suitable material plus perhaps other additives. Although these are the main ingredients other items may be added. It can be used as a spread or topping or be frozen so as to present as a popsicle or frozen dessert or as part of a spread or topping or frozen product.

Basic:

One particular recipe is for one volume of grape seed flour (could further substitute other high antioxidant natural powders) to one of coconut oil heated for three minutes and then up to two to four volumes of prepared red bean paste with sweetener for further flavor and mix or cook on low heat while stirring or blending for another 5 minutes. Could serve this at room temperature or warm on toast or cracker or encased in rice flour or other cooked flour in a desert or on fruit or as a frozen desert such as a popsicle or in a capsule.

Another particular recipe is to encapsulate grape seed flour (or other high antioxidant powders) or grape seed extract with high MCT oil or coconut oil.

Prior to this invention these flours had not been combined with high MCT oils or coconut oil for food or supplement consumption.

Purpose is to provide a palatable form of natural antioxidants which will be a health improving and acceptable even to those resistant to vegetable or fruit consumption or supplementation.

A further purpose is to provide the same combinations of oil and powder in capsules as supplements although the concentrations could be higher for the powder in the oil, especially in coconut oil. By providing an oil “carrier” with the powder the amount of absorption of the antioxidants is improved by presenting the antioxidants to different parts of the digestive system as much of the antioxidant strong powder is carried through the stomach to the small and large intestines.

A further purpose is to provide the antioxidant powder in suspension of coconut oil so that lipid soluble antioxidants can be preabsorbed and then presented to the digestion separately than the water soluble antioxidants which will be initially protected in the powder from digestion by the coating of the high MCT or coconut oil and then digested further into the digestive system as the oil is fully taken up and exposes the particles to the digestive system. Thus the oil and powder act synergistically as the high antioxidant powder is presented to a more extensive portion of the digestive system.

### REFERENCES

[1] Rui Hai Liu. Health benefits of fruit and vegetables are from additive and synergistic combinations of phytochemicals. Am J Clin Nutr September 2003 vol. 78 no. 3 517S-520S.
[2] Augustin Scalbert, Claudine Manach, Christine Morand, Christian Rémésy & Liliana Jiménez. Dietary Polyphenols and the Prevention of Diseases. Critical Reviews in Food Science and Nutrition. Volume 45, Issue 4, 2005.
[3] Yao L H¹, Jiang Y M, Shi J, Tomás-Barberán F A, Datta N, Singanusong R, Chen S S. Flavonoids in Food and their Health Benefits. Plant Foods Hum Nutr. 2004 Summer; 59(3):113-22.
[4] Ana García-Lafuente, Eva Guillamóm, Ana Villares, Mauricio A. Rostagno, José Alfredo Martínez. Flavonoids as anti-inflammatory agents: implications in cancer and cardiovascular disease. Inflammation Research. September 2009, Volume 58, Issue 9, pp 537-552
[5]. Hartman Research Group Newsletter. Hartbeat—Monitoring the Rhythm of Consumer Culture. Who Put The Function In Functional Foods? 2005 Jun. 22
[6]. Academy of Nutrition and Dietetics. Position of the Academy of Nutrition and Dietetics: Functional Food. Volume 113, Number 8 pages 1096-1103 (August 2013)
[7],[9],[10]. Natasha Singer. ‘Foods With Benefits, or So They Say’. The New York Times. May 14, 2011
[8]. Jennifer K. Nelson, R. D. L. D. I've Heard the Term “Functional Foods”, but I Don't Know What it Means. Can You Explain? Mayo Clinic Newsletter—Lifestyle. Jun. 9, 2012
[11]. Liwei Gu, Mark A. Kelm*, John F. Hammerstone*, Gary Beecher^{†}, Joanne Holden^{†}, David Haytowitz^{†}, Susan Gebhardt^{†}, and Ronald L. Prior³ Concentrations of Proanthocyanidins in Common Foods and Estimations of Normal Consumption. J. Nutr. Mar. 1, 2004 vol. 134 no. 3 613-617
[12] Mojca \check{S}kerget^{a}, Petra Kotnik^{a}, Majda Hadolin, Andreja Ri\check{z}ner Hra\check{s}, Marjana Simoni\check{c}, \check{Z}eljko Knez. Phenols, proanthocyanidins, flavones and flavonols in some plant materials and their antioxidant activities. Food Chemistry Volume 89, Issue 2, February 2005, Pages 191-198
[13]. Schwitters, Bert (1995) OPC in Practice. Publishing rights search incomplete p. 15 ISBN 88-86035-13-6
[14] Corder, R.; Mullen, W.; Khan, N. Q.; Marks, S. C.; Wood, E. G.; Carrier, M. J.; Crozier, A. (2006). “Oenology: Red wine procyanidins and vascular health”. Nature 444 (7119): 566. doi:10.1038/444566a. PMID 17136085.
[15] Shi, John; Yu, Jianmel; Pohorly, Joseph E.; Kakuda, Yukio (2003). “Polyphenolics in Grape Seeds Biochemistry and Functionality”. Journal of Medicinal Food 6 (4): 291 doi:10.1089/109662003772519831. PMID 14977436.
[16] Xu Y, Li S, Chen R et al. (January 2010). “Antidepressant-like effect of low molecular proanthocyanidin in mice: involvement of monoaminergic system”. Pharmacol. Biochem. Behav. 94 (3): 447-53. doi:10.1016/j.pbb.2009.10.007. PMID 19857512.
[17] Robert A Koeth, Zeneng Wang, Bruce S Levison, Jennifer A Buffa, Elin Org, Brendan T Sheehy, Earl B Britt, Xiaoming Fu, Yuping Wu, Lin Li, Jonathan D Smith, Joseph A DiDonato, Jun Chen, Hongzhe Li, Gary D Wu, James D Lewis, Manya Warrier, J Mark Brown, Ronald M Krauss, W H Wilson Tang, Frederic D Bushman, Aldons J Lusis & Stanley L Hazen. “Intestinal microbiota metabolism of L-carnitine, a nutrient in red meat, promotes atherosclerosis.” The Journal of Nature Medicine. 7 Apr. 2013: 576-585
[18}. Newport, Mary T., M. D., The Story of Keytones. Laguna Beach, Califorina: Basic Health Publications, Inc., 2011
[19] Wang Y J, Thomas P, Zhong J H, Bi F F, Kosaraju S, Pollard A, Fenech M, Zhou X F Consumption of grape seed extract prevents amyloid-beta deposition and attenuates inflammation in brain of an Alzheimer's disease mouse. Article on Grape Seed Extract from Flinders University in South Australia: (Neurotox Res. 2009 January; 15(1):3-14. doi: 10.1007/s12640-009-9000-x. Epub 2009 Feb. 10.]
[20] University of Maryland Medical Center. Alzheimer's Disease. University of Maryland Medical Center Newsletter. May 7, 2013 http://umm.edu/health/medical/reports/articles/alzheimers-disease
[21] Zhao Y, Zhao B. Natural antioxidants in prevention and management of Alzheimer's disease. Front Biosci (Elite Ed). 2012 Jan. 1; 4:794-808. Review.
[22] Darvesh A S¹, Carroll R T, Bishayee A, Geldenhuys W J, Van der Schyf C J. Oxidative stress and Alzheimer's disease: dietary polyphenols as potential therapeutic agents. Expert Rev Neurother. 2010 May; 10(5):729-45. doi: 10.1586/ern.10.42.

I claim:

1. Functional foods consisting at least of combinations of very high antioxidant powders and a high MCT oil, with the purpose of providing both the high antioxidants and the MCTs, which both have extensive nutritional benefits separately and synergistically.

2. The functional foods of claim 1 wherein the oil is a fraction of coconut oil such as the MCT's.

3. The functional foods of claim 1 wherein one high antioxidant powder includes Grape Seed Flour, containing proanthocyanidins.

4. The functional Foods of claim 1 wherein one high antioxidant powder is made from goji berries.

5. The functional foods of claim 1 wherein the high antioxidant powder is made from matcha.

6. The functional foods of claim 1 wherein the high antioxidant powder is made from cocoa powder.

7. The functional foods of claim 1 wherein the high antioxidant powder is Achai fruit and skin pulp powder

8. The functional foods of claim 1 wherein high antioxidant powder is cinnamon powder.

9. the functional foods of claim 1 wherein one high antioxidant powder is turmeric

10. the functional foods of claim 1 wherein one high antioxidant powder is maqui berry.

11. the functional foods of claim 1 wherein one high antioxidant powder is sorghum bran

12. A supplement using the combination of a high antioxidant powder with high MCT oil in a capsule.

13. The supplement of claim 12 including grape seed powder with high MCT oil in a capsule.

14. The supplement of claim 12 using ginseng powder with high MCT oil in a capsule.

15. The supplement of claim 12 using cinnamon powder with high MCT oil in a capsule

16. The supplement of claim 12 using turmeric powder with high MCT oil in a capsule.

17. Functional foods using the combination of the ingredients in claim 1 plus a paste of beans so as to make the taste more acceptable and applicable to a wider range of foods.

18. The functional foods in claim 17 wherein the powder used is grape seed flour and the paste is made with Adzuki beans.

19. As in claim 17 but presented in a frozen product. This represents a new use for Grape Seed flour combined with Red Bean paste.

20. As in claim 17 but presented as a topping. This represents a new use for both Grape Seed flour and for Red Bean paste.

21. A supplement using a combination of any extract powder perhaps normally presented as a tablet or encapsulated dry or taken as a powder, instead encapsulated in an oil. It is therefore a form of micro-encapsulation of the fine particles

22. The supplement in claim 21 using any extract substance powdered and encapsulated with a high MCT oil.
